(19) United States
(12) Patent Application Publication
(10) Pub. No.: US 2024/0049760 A1
(43) Pub. Date: Feb. 15, 2024

(54) SAFE-TO-INGEST MICROEMULSIONS AND NANOEMULSIONS CONTAINING LIPOPHILIC COMPONENTS

(71) Applicant: Petro Pawlo Czupiel, Toronto (CA)

(72) Inventor: Petro Pawlo Czupiel, Toronto (CA)

(21) Appl. No.: 18/259,176

(22) PCT Filed: Dec. 24, 2021
(86) PCT No.: PCT/IB2021/062309

§ 371 Date: Jun. 23, 2023

Related U.S. Application Data
(60) Provisional application No. 63/199,418, filed on Dec. 24, 2020.

(51) Int. Cl. A23L 29/10; A23L 33/105; A23L 33/115

(52) U.S. Cl. CPC A23L 29/10; A23L 33/105; A23L 33/115

(57) ABSTRACT

The present invention is directed toward compositions and methods of use of cationic microemulsions and nanoemulsions of lipophilic components. More particularly, the present invention pertains to formulating stable cationic microemulsions and nanoemulsions that have the potential to better interact with human cells, relative to standard microemulsions and nanoemulsions.

### FIELD OF THE INVENTION

The present invention relates generally to emulsions of lipophilic components and more specifically to compositions and methods for preparation of ingestible emulsions containing lipophilic components.

### BACKGROUND

Drinking water and eating food products is an essential method to deliver nutrients and other molecules that are water-soluble into the body. Nutrients and other molecules that are partially soluble or insoluble in water, termed lipophiles, that are essential to a good health demonstrate significant difficulty in entering the blood circulation when introduced to the body, and hence have poor bioavailability. The poor bioavailability of lipophiles is worsened as they are not absorbed by the gastrointestinal tract and is amplified in end users with malabsorption conditions. Lipophiles can range from a non-exhaustive list of insoluble vitamins, such as Vitamin D, dietary supplements, such as coenzyme Q10, psychoactive agents, such as THC, analgesic agents, such as CBD, fatty acids, such as omega 3 and omega 6, and essential oils among others.

There is a need to include lipophiles into water-based formulations, such as beverages and supplements, to increase the bioavailability of the lipophile. Water-based formulations are more preferred for end users who are experiencing “pill fatigue,” in which the end user is tired of consuming multiple pills or capsules in addition to users who have trouble swallowing pills or capsules. Moreover, it is known that human cells exhibit a negatively charged plasma membrane of approximately −60 mV, that presents an electrostatically attractive property when preparing water-based formulations. Furthermore, formulations taken orally and passed through the stomach and then liver experience a first pass effect, whereby more than 80% of the nutrient or lipophile is retained in the liver. It is desirable to prepare formulations that can be held under the tongue and the nasopharynx, to minimize liver retention. Therefore, there is a need to formulate lipophiles into a water-based formulation that may exploit the negatively charged plasma membranes found throughout the body. Importantly, these water-based formulations should present desirable organoleptic properties such that the end user favors the taste, smell, and overall perception of the final formulation.

Some known prior art teaches that water-soluble technologies exist for the solubilization of anti-cancer pharmaceuticals and other lipophiles. Specifically, prior art on water-soluble technologies focus on nanocapsules, nanospheres, liposomes, emulsions, microemulsions and nanoemulsions where the diameters of the various technologies can vary from 10 nm-1,000,000 nm. It is desirable to formulate water-soluble technologies with smaller diameters, such that they circulate throughout the body for an extended time as evidenced in the past.

With the changing world perception on cannabis, cannabis products have gained a significant amount of detail. Cannabis contains more than 460 compounds, of which tetrahydrocannabinol (THC), cannabidiol (CBD), beta-caryophyllene and other cannabinoids and terpenes that can interact with CB1 and CB2 receptors. These interactions are then followed by therapeutic effects such as psychoactive perception, analgesia, calmness, anti-inflammatory, antiemetic, antispastic and anti-anxiety. Furthermore, cannabis has also been demonstrated to decrease epilepsy. Therefore, molecules found within cannabis can illicit multiple and beneficial therapeutic effects.

Cannabis is typically heated by flame or oven while smoking or vaping and the plethora of compounds enter the blood stream in the lungs where the psychoactive and/or analgesic effects are felt within minutes. The bioavailability of cannabinoids following smoking or vaping has been reported to be 2-56%, dependent on intra- and inter-subject variability. Smoking or vaping is typically viewed as unhealthy and damaging to the lungs, where healthier alternatives are being sought out by end users. One such healthy alternative includes the ingestion of oils, capsules, or pills containing cannabinoids. Unfortunately, cannabinoids are sparingly soluble in water as they are lipophiles and must be dissolved in oils for oral administration. Bioavailability following oral ingestion of cannabinoid oils is typically less than 6%. Following oral administration, end users typically feel the psychoactive and/or analgesic effects 90-120 minutes after ingestion of the cannabinoid containing oil. The ingestible oil route can be further enhanced by encapsulation in microemulsions or nanoemulsions and, therefore, there is a need to prepare water-soluble formulations of cannabinoids.

### SUMMARY

Cationic microemulsion and nanoemulsion compositions are provided that contain lipophilic components, such as cannabinoids and terpenes, that are safely ingested. Also provided are methods to manufacture the cationic microemulsion and cationic nanoemulsion. Also provided are liquid dilution compositions for methods to dilute the cationic microemulsion and cationic nanoemulsion into the desired parameters for a consumer-packaged good product.

Thus by one broad aspect of the present disclosure, a cationic emulsion composition is provided that includes at least one lipophile in an amount between 0.01% and 10% by weight (w/w) of the composition, an ingestible emulsifier in an amount between 1% and 40% w/w, an ingestible cationic emulsifier in an amount between 0.01% and 10%, an ingestible oil in an amount between 0.01% and 5%, an ingestible pH adjuster in an amount between 0.001% and 5%, an ingestible preservative in an amount between 0.0001% and 1%, an ingestible co-solvent in an amount between 0.10% and 50%, a sequestering agent in an amount between 0.001% and 1%; and an ingestible sweetener in an amount between 0.001% and 2%.

By a further broad aspect of the present disclosure, a method for manufacture of a cationic microemulsion and a cationic nanoemulsion is provided, including weighing the components of an emulsion composition, heating the combined emulsion composition from about 25° C. to about 95° C. with agitation from a rotor/stator to prepare the emulsion, and cooling the microemulsion composition to about 25° C.

### DETAILED DESCRIPTION

Unless specifically noted otherwise herein, the definitions of the terms used herein are standard definitions used in the art of organic synthesis and pharmaceutical sciences. Exemplary embodiments, aspects and variations are illustrative in the examples, and it is intended that the embodiments, aspects and variations, and the figures and drawings disclosed herein are to be considered illustrative and not limiting.

As used herein, the singular forms “a,” “an” and “the” include plural referents unless the context clearly dictates otherwise. Thus, for example, reference to compound, comprising “an extracellular domain” includes compounds with one or a plurality of extracellular domains.

As used herein, ranges and amounts can be expressed as “about” a particular value or range. About also includes the exact amount. Hence “about 10 w/w %” means “about 10 w/w %” and also “10 w/w %.” It also is understood that ranges expressed herein include whole numbers within the ranges and fractions thereof. For example, a range of between 10 w/w % and 20 w/w % includes whole number values such as 10, 11, 12, 13, 14, 15, 16, 17, 18, 19 and 20 w/w %, and fractions within the range, for example, but not limited to, 10.25, 16.72, 18.5, and 19.95 w/w %.

As used herein, the term “emulsifier,” “ingestible cationic emulsifier,” and “ingestible emulsifier” refer synonymously to ingredients that form and stabilize colloidal dispersions of two immiscible liquids, for example, an oil and water.

Provided are cationic microemulsion and nanoemulsion compositions that contain lipophilic components, such as cannabinoids and terpenes, that are compliant with governmental regulations to be safely ingested. Also provided are methods to manufacture the cationic microemulsion and cationic nanoemulsion. Also provided are liquid dilution compositions for methods to dilute the cationic microemulsion and cationic nanoemulsion into the desired parameters for the consumer-packaged good product. The cationic nanoemulsions or cationic microemulsions may also incorporate one or more lipophilic components selected from the group consisting of other cannabinoids, terpenes, antioxidants, fat soluble vitamins, fatty acids, carotenoids, hormones, metabolic factors, phytochemicals, phytonutrients, phytosterols, vitamin derivatives and other oil-based lipophilic components. The cationic nanoemulsions or cationic microemulsions provided herein are stable emulsions without creaming, flocculation, or phase separation.

The cationic microemulsions contain lipophilic components in an amount between at or about 0.01% and at or about 10.00%; and emulsifiers such as polysorbate 80 alone or in combination with tocopheryl polyethylene glycol succinate present in an amount between at or about 10% and at or about 40%; and cationic emulsifiers such as ethyl lauroyl arginate present in an amount between at or about 0.01% and at or about 10.00%; and ingestible oils such as extra virgin olive oil present in an amount between at or about 0.01% and at or about 5.00%; and pH adjuster such as citric acid present in an amount between at or about 0.001% and at or about 5.000%; and preservatives such as sodium benzoate alone or in combination with potassium sorbate present in an amount between at or about 0.001% and at or about 1.000%; and cosolvents such as glycerin alone or in combination with propylene glycol or ethanol is present in an amount between at or about 0.1% and at or about 30.0%; and sequestering agent selected from the group consisting of ethylenediaminetetraacetic acid (EDTA), calcium disodium EDTA, disodium EDTA and mixtures thereof is present in an amount between at or about 0.001% and at or about 1.000%; and an ingestible sweetener such as sucralose is present in an amount between at or about 0.001% and at or about 2.000%, inclusive, by weight, of the composition.

The previously described cationic microemulsion can be alternatively modified to add or to replace the tocopheryl polyethylene glycol succinate with one or more of the emulsifiers selected from the group consisting of polysorbate 60, macrogolglycerol hydroxystearate such as Kolliphor RH40, and macrogolglycerol ricinoleate such as Kolliphor EL, and combinations thereof, in an amount between at or about 5% and at or about 30%, inclusive, by weight, of the composition.

The cationic nanoemulsions contain emulsifiers such as polysorbate 80 alone or in combination with lecithin present in an amount between at or about 1% and at or about 10%; and cationic emulsifiers such as ethyl lauroyl arginate present in an amount between at or about 0.010% and at or about 10.00%; and ingestible oils such as extra virgin olive oil present in an amount between at or about 0.01% and at or about 5.00%; and pH adjuster such as citric acid present in an amount between at or about 0.001% and at or about 5.000%; and preservatives such as sodium benzoate alone or in combination with potassium sorbate present in an amount between at or about 0.0010% and at or about 1.000%; and cosolvents such as glycerin alone or in combination with propylene glycol or ethanol is present in an amount between at or about 0.1% and at or about 10.0%; and sequestering agent selected from the group consisting of ethylenediaminetetraacetic acid (EDTA), calcium disodium EDTA, disodium EDTA and mixtures thereof is present in an amount between at or about 0.001% and at or about 1.000%; and an ingestible sweetener such as sucralose is present in an amount between at or about 0.0010% and at or about 2.000%, inclusive, by weight, of the composition.

The previously described cationic nanoemulsion can be alternatively modified to add or to replace the tocopheryl polyethylene glycol succinate with one or more of the emulsifiers selected from the group consisting of polysorbate 60, macrogolglycerol hydroxystearate such as Kolliphor RH40, and macrogolglycerol ricinoleate such as Kolliphor EL, and combinations thereof, in an amount between at or about 5% and at or about 30%, inclusive, by weight, of the composition. The previously described cationic nanoemulsion can be further modified comprising one or more ingestible emulsifiers that do not contain polyoxyethylene selected from the group consisting of sucrose monolaurate, sucrose monopalmitate, sucrose monostearate, span 20, span 40, span 60, or a combination thereof present in an amount between at or about 0.1% and at or about 5%, inclusive, by weight, of the composition. The previously described cationic nanoemulsions further comprising one or more polymer emulsifiers selected from the group consisting of Acacia gum, Acacia Gum modified with octenyl succinic anhydride, acetylated monoglycerides, agar, algin, alginic acid, ammonium carrageenan, Arabino-galactan, calium alginate, calcium carrageenan, carboxymethyl cellulose, carob bean gum, carrageenan, cellulose gum, gellan gum, guar gum, gum Arabic, hydroxylated lecithin, hydroxypropyl cellulose, hydroxypropyl methylcellulose, Karaya gum, Lactylated Mono- and Di-glycerides, lactylic esters of fatty acids, locust bean gum, methyl cellulose, methyl ethyl cellulose, monoglycerides, mono- and di-glycerides, oat gum, pectin, polyglycerol esters of fatty acids, polyglycerol esters of interesterified castor oil fatty acids, polyoxyethylene (8) stearate, potassium alginate, potassium carrageenan, propylene glycol alginate, propylene glycol ether of methylcellulose, Quillaia extract, sodium alginate, sodium carboxymethyl cellulose, sodium carrageenan, sodium cellulose glycolate, sodium stearoyl-2-lactylate, tamarind gum, tara gum, tragacanth gum, xanthan gum, or a combination thereof present in an amount between at or about 0.01% and at or about 1%, inclusive, by weight, of the composition.

The previously described cationic microemulsions and cationic nanoemulsions further comprising bicarbonate or carbonate present in an amount between at or about 0.001% and at or about 5%, inclusive, by weight, of the composition.

The previously described cationic microemulsions and cationic nanoemulsions further comprising one or more vegetable oils selected from the group consisting of medium-chain triglycerides (MCLs), short-chain triglycerides, glyceryl monooleate, glyceryl monolinoleate, polyglyceryl 3-oleate, polyglyceryl 4-oleate, glyceryl palmitate, glyceryl palmitostearate, glyceryl ricinoleate, glyceryl esters of saturated fatty acids, glyceryl behenate, polyglyceryl 1O-oleate, polyglyceryl 1O-tetralinoleate, behenic acid, caprylyic/capric glycerides sesame oil or coconut oil and a mineral oil, cannabis oil (hemp oil), coconut oil, cottonseed oil, soybean oil, glyceryl distearate, glyceryl isostearate, glyceryl aurate, glyceryl stearate, almond oil, sesame oil, rapeseed oil, peppermint oil, canola oil, palm kernel oil, arachis oil, olive oil, sunflower oil, castor oil, corn oil, olive oil, palm oil, peanut oil, hydrogenated soybean oil, and combinations thereof.

The previously described cationic microemulsions and cationic nanoemulsions further comprising one or more masking or flavoring components selected from the group consisting of thyme oil, artificial, natural or synthetic fruit flavors selected from the group consisting of vanilla, chocolate, natural cinnamon oil, peppermint oil, coffee, cocoa, and citrus oil, clove oil, bay oil, selected from the group consisting of grape, grapefruit, lemon, lime, orange, and fruit essences selected from the group consisting of cherry, plum, watermelon, raspberry, peach, strawberry, apple, pear, pineapple and apricot, or combinations thereof.

The previously described cationic microemulsions and cationic nanoemulsions further comprising a reducing agent selected from the group consisting of vitamin C, ubiquinol, L-ascorbic acid-8-palmitate, or mixtures thereof.

The cannabis oil or distillate used for the cationic microemulsion or cationic nanoemulsions comprise 10% or less, by weight, of the composition. The cannabis oil or distillate may contain cannabinoids such as cannabis oil or distillate containing cannabigerol (CBG), cannabichromene (CBC), cannabidiol (CBD), Δ8-tetrahydrocannabinol, Δ9-tetrahydrocannabinol (THC), cannabicyclol (CBL), cannabielsoin (CBE), cannabinol (CBN), cannabinodiol (CBDL), cannabitriol (CBTL), cannabichromene, tetrahydrocannabivarin, tetrahydrocannabinolic acid, cannabidivarin, cannabidiolic acid, cannabigerol monomethyl ether or mixture thereof; and terpenes such as β-Myrcene, D-Limonene, β-Ocimene, γ-Terpinene, α-Terpinene, α-Terpineol, α-Pinene, β-Pinene, Linalool, Camphene, Terpinolene, α-Phellandrene, γ-Cadinene, Δ3-Carene, ρ-Cymene, fenchol, 1,8-cineole (eucalyptol), O-Caryophyllene, Caryophyllene Oxide, Humulene, (α-Caryophyllene), β-Elemene, Guaiol, Eudesmol Isomers, Nerolidol, Gurjunene, γ-Cadinene, β-Famesene, carvone, taxadiene, squalene, geraniol, geranylfamesol, lycopene or mixtures thereof.

The previously described cationic microemulsions and cationic nanoemulsions are clear and do not contain visible particles or precipitants with a Nephelometric Turbidity Units (NTU) value of less than 200, or about 200, less than 100 or about 100. The previously described cationic microemulsions and cationic nanoemulsions comprise of particle size that are less than 150 nm; or between 10 and 150 nm, as measured by dynamic light scattering equipment. The previously described cationic microemulsions and cationic nanoemulsions comprise a zeta-potential between at or about +10 millivolts (mV) and at or about +60 millivolts, inclusive, as measured by a electrophoretic mobility apparatus, such as a Zetasizer Nano Z (Malvern Instruments Ltd, United Kingdom). The non-ionic emulsifiers that contain polyoxyethylene and may be incorporated in the nanoemulsions include, but are not limited to: Octaethylene glycol monododecyl ether, pentaethylene glycol monododecyl ether, triton x-100, polyethoxylated tallow amine, polyethylene glycol nonyl phenyl ether, cocamide diethanoloamine, poloxamers, glycerol monostearate, glycerol monolaurate, sorbitan monolaurate, sorbitan monostearate, sorbitan tristate, polysorbate 20, polysorbate 40, polysorbate 60, polysorbate 80, fatty acid esters of sucrose, sucrose monolaurate, sucrose monopalmitate, surcrose monostearate, decyl glucoside, lauryl glucoside, octyl glucoside, lauryldimethylamine oxide, sorbitan esters such as span 20, span 60, span 65, arlacel 165, arlacel 170, ethoxylated 25 alcohols such as Brij, Tergitol NP-40 and NP-70, polysorbates, polyoxyethanyl-cholesterol-sebacate (PCS), polyoxyethanyl-tocopheryl-sebacate, polyoxyethanyl-ubiquinol-sebacate polyoxy ethanyl-sitosterol-sebacate, D-α-Tocopherol polyethylene glycol 1000 succinate (TPGS) and related polyethylene glycol modified tocopheryl derivatives. Zwitterionic surfactants may be used in combination with non-ionic surfactants such as (3-[(3-cholamidopropyl)dimethylammonio]-1-propanesulfonate) (CHAPS), cocamidopropyl hydroxusultaine, cocamidopropyl betaine, phosphatidylserine, phosphatidylethanolamine, phosphatidylcholine, sphingomyelins. Anionic surfactants may also be combined with the non-ionic surfactants such as docusate, alkyl-aryl ether phosphate, alkyl ether phosphate.

The cationic emulsifiers that may be encapsulated in the nanoemulsions include, but are not limited to: ethyl lauroyl arginate, benzalkonium chloride, benzethonium chloride (BZT), cetrimonium bromide (CTAB), cetylpyridinum chloride (CPC), octenidine dihydrochloride, dimethyldioctadecylammonium chloride, dioctadecyldimethylammonium bromide (DODAB), cetyltrimethyl ammonium chloride.

The polymer emulsifiers that may be incorporated for topical administration may also include, but are not limited to: Acacia gum, Acacia Gum modified with octenyl succinic anhydride, acetylated monoglycerides, agar, algin, alginic acid, ammonium carrageenan, Arabino-galactan, calium alginate, calcium carrageenan, carboxymethyl cellulose, carob bean gum, carrageenan, cellulose gum, gellan gum, guar gum, gum Arabic, hydroxylated lecithin, hydroxypropyl cellulose, hydroxypropyl methylcellulose, Karaya gum, Lactylated Mono- and Di-glycerides, lactylic esters of fatty acids, locust bean gum, methyl cellulose, methyl ethyl cellulose, monoglycerides, mono- and di-glycerides, oat gum, pectin, polyglycerol esters of fatty acids, polyglycerol esters of interesterified castor oil fatty acids, polyoxyethylene (8) stearate, potassium alginate, potassium carrageenan, propylene glycol alginate, propylene glycol ether of methylcellulose, Quillaia extract, sodium alginate, sodium carboxymethyl cellulose, sodium carrageenan, sodium cellulose glycolate, sodium stearoyl-2-lactylate, tamarind gum, tara gum, tragacanth gum, xanthan gum.

The oils that may be used in the nanoemulsions include, but are not limited to: Amaranth oil, apricot oil, apple seed oil, argan oil, avocado oil, Agai oil, Almond oil, beech nut oil, babassu oil, Brazil nut oil, bitter gourd oil, bottle gourd oil, ben oil, bomeo tallow nut oil, black seed oil, blackcurrent seed oil, borage seed oil, butternut squash seed oil, cape chestnut oil, carob pod oil, cocoa butter, cocklebur oil, cohune oil, coriander seed oil, cashew oil, canola oil, coconut oil, corn oil, cottonseed oil, date seed oil, dika oil, extra virgin olive oil, eugsi seed oil, evening primrose oil, false flax oil, flaxseed oil, grape seed oil, grapefruit seed oil, hazelnut oil, hemp oil, kapok seed oil, kenaf seed oil, lallemantia oil, lemon oil, macadamia oil, mongongo oil, marula oil, meadowfoam seed oil, mustard oil, niger seed oil, nutmeg butter, orange oil, okra seed oil, olive oil, papaya seed oil, persimmon seed oil, pequi oil, pilinut oil, pomegranate seed oil, poppyseed oil, pracaxi oil, prune kernel oil, seed oil perilla seed oil, palm oil, peanut oil, pecan oil, pine nut oil, pistachio oil, pumpkin seed oil, quinoa oil, ramtil oil, rice bran oil, royle oil, rapeseed oil, safflower oil, sacha inchi oil, sapote oil, seje oil, shea butter, sesame oil, soybean oil, Extra Virgin Olive Oil, taramira oil, tea seed oil, thistle oil, tigernut oil, tobacco seed oil, tomato seed oil, virgin olive oil, walnut oil, watermelon seed oil, wheat germ oil.

The lipophiles that may be encapsulated in the nanoemulsions include, but are not limited to: Antioxidants (alpha-lipoic acid, R-lipoic acid, carotenoids, catechins, catalase, epicatechins, flavonoids, glutathione, SOD); fat Soluble Vitamins (vitamins A, D, E (alpha-tocopherol, mixed tocopherols, tocotrienols), K1, K2); fatty Acids (CLA, DHA, EPA, GLA, docosahexanoic acid, marine lipids, omega-3 and omega-6 fatty acids, blackcurrant seed oil, borage oil, evening primrose oil, hemp seed oil, cetyl myristoleate, conjugated linoleic acid, flax seed oil, phosphatidyserine, krill oil, fumaric acid, undecylenic acid, shark liver oil; carotenoids (beta-carotene, lutein, luteolin, lycopene, staxanthin, zeaxanthin); hormones (pregnenolone, melatonin, 7-Ketodehydroepiandrosterone (7-Keo DHEA), dehydroepiandrosterone (DHEA)); metabolic Factors (5-hydroxy tryptophan(5-HTP), beta-phenylethylamine, Coenzyme Q10, ferulic acid, forskolin, gamma oryzanol, L-tyrosine, lipoic acid, N-acetyl tyrosine, phenibut, piracetam, serapeptidase); Cannabinoids such as cannabigerol (CBG), cannabichromene (CBC), cannabidiol (CBD), Δ⁸-tetrahydrocannabinol, Δ⁹-tetrahydrocannabinol (THC), cannabicyclol (CBL), cannabielsoin (CBE), cannabinol (CBN), cannabinodiol (CBDL) and cannabitriol (CBTL) containing cannabigerol (CBG), cannabichromene (CBC), cannabidiol (CBD), Δ8-tetrahydrocannabinol, Δ 9-tetrahydrocannabinol (THC), cannabicyclol (CBL), cannabielsoin (CBE), cannabinol (CBN), cannabinodiol (CBDL), cannabitriol (CBTL), cannabichromene, tetrahydrocannabivarin, tetrahydrocannabinolic acid, cannabidivarin, cannabidiolic acid, cannabigerol monomethyl ether or mixture thereof; Terpenes such as β-Myrcene, D-Limonene, β-Ocimene, γ-Terpinene, α-Terpinene, α-Terpineol, α-Pinene, β-Pinene, Linalool, Camphene, Terpinolene, α-Phellandrene, γ-adinene, Δ3-Carene, ρ-Cymene, fenchol, 1,8-cineole (eucalyptol), β-Caryophyllene, Caryophyllene Oxide, Humulene, (α-Caryophyllene), β-Elemene, Guaiol, Eudesmol Isomers, Nerolidol, Gurjunene, γ-Cadinene, β-Famesene, carvone, taxadiene, squalene, geraniol, geranylfarnesol, lycopene; phytochemicals (20-hydroxy ecdysterone, 4-(4-hydroxyphenyl)-2-butanone, berberine, caffeine, chrysin, curcumin, dihydromyrecetin, diindolylmethane (DIM), daidzein, ellagic acid, forskolin, fucoxanthin, glalantamine, genistein, guggul lipid components, glycyrrhizic acid, hesperidin methyl chalcone, hordenine, huperzine a, huperzine, icariin, maslinic acid, myrecetin, natokinase, osthole, policosanol, pterostilbene, pyrroloquinoline quinone (PQQ), quercetin, resveratrol, red wine poyphenols, red yeast rice, rutin, silymarin, vinpocetine); phytonutrients (ajugatukestanica, artichoke extract, araliamandshuricaextract, andrographis, angelica sinensis extract, ajuga turkestanica, algae extract, astragulus extract, ashwagandha extract, black cohosh extract, cordyceps extract, bitter melon extract, bitter melon fruit extract, bitter melon extract, bilbery extract, blue corn color, boswellin, cnidium extract, cranberry extract, cramp bark extract, cats claw extract, centella asiatica extract, corioius vesicolor extract, cinnamon extract, cranberry extract, citrus seed extract, curcumin, dandelion root extract, eurcoma longifolia jack extract, epimedium extract, garcinia cambogia extract, ginger extract, Ginkgo biloba extract, green tea extract, green coffee bean extract, ganoderma extract, grape seed extract, gymnema sylvestre leaf extract, hawthorn berry extract, hawthorn extract, horsetail extract, haritaki extract, irvingia gabonensis extract, Japanese knotwood extract, kola nut extract, licorice root extract, lentinula adode extract, muira puama bark extract, maca root extract, miura puama extract, Momordica charantia extract, maitake extract, mushroom extracts, olive leaf extract, pasiflora incarnata extract, Panax ginseng extract, pygeum extract, pine bark extract, Prunus africanus extract, pomegranate extract, pau d'arco extract, Ruscus aculeatus extract, rosemary extract, rhaponticum cathaomids extract, Rhodiola rosea extract, St. John's Wort extract, saw palmetto fruit extract, siberian ginseng root extract, sambaia extract, silymarin, schizandra berry extract, somnifera extract, stinging nettles extract tumeric extract, Tribulus terrestris, urtica dioca extract, Uva ursi leaf extract, valerian extract, Vitex agnus castus extract, withania, yerba mate extract,); phytosterols (β-Sitosterol, stigmasterol); vitamin derivatives (benfotiamine).

The cooling method in the provided examples use ice as the cooling agents but other methods can be utilized such as cooling heat exchangers, industrial heat exchangers, near freezing temperature water.

The following examples are provided for illustrative purposes only and are not intended to limit the scope of the invention. A list of the examples can be found below:
a. Example 1: Cationic microemulsions using ethyl lauroyl arginate
b. Example 2: Cationic nanoemulsions using ethyl lauroyl arginate

### Example 1: Cationic Microemulsions Using Ethyl Lauroyl Arginate

Table 1A below depicts the microemulsion composition ranges that are covered by this disclosure. The label for “any lipophile” may include either one lipophile or a combination of two or more lipophiles as previously described. Table 1B depicts the microemulsion compositions of the ingredients required to make a 50 kg batch of cationic microemulsions containing 1.6% of cannabis distillate with 80% purity for THC. It should be noted that the ingredients in the microemulsion are added with an additional 5 weight % overage. Water is described as sterile distilled water. The cannabis distillate may be obtained from various standard processing pathways obtained through either supercritical or subcritical carbon dioxide extraction. Cannabis distillate may also be replaced with cannabis extract obtained from ethanol, propane, or butane extraction. Exemplary food grade TPGS was sourced from Antares Health Product, Inc. Exemplary food grade Polysorbate 80 was sourced from Spectrum Chemical Mfg Corp (product number: PO138, Cas. Number: 9005-65-6). Exemplary food grade ethyl lauroyl arginate was sourced from Vedeqsa (Product name: Miranat-G, Product code: 3407). Exemplary food grade sunflower oil is sourced from Jedwards Internation Inc (SKU: S1630). Sugar alcohol, preservatives, sugars, sweeteners and flavoring agents may be added to each table as desired in the finished consumer packaged good product.

Table 1A below sets forth ingredient compositions that would be compatible with the preparation of the microemulsions. Table 1B sets forth ingredients that are required for a 50 kg batch of master stock microemulsion 1B containing either TPGS or polysorbate 80, and ethyl lauroyl arginate. This assumes that the cannabis distillate has 80% purity for THC and that targeted THC concentration is 12.8 mg/mL. The ingredients are added into a suitable stainless-steel hot liquid tank. Exemplary of the tanks that can be used with the provided methods are water-jacketed tanks, for example, the Camak 42-gallon water-jacketed tank (Model: AGM 90 S 4). An appropriate scale is used to accurately measure the mass of each ingredient being added into the water-jacketed tank. The following ingredients are added in sequential order: 1) Cannabis distillate, 2) TPGS or polysorbate 80, 4) ethyl lauroyl arginate, 5) sunflower oil, and 7) water. The water is then heated to 90° C. (±5° C.) and the batch is mixed at 400 RPM for 30 minutes. The temperature was monitored with a suitable temperature meter, for example, Cooper-Atkins temperature probe (Model: DPP400W). After the intended mixing and heating time, the microemulsion was cooled down to 25° C. (±5° C.). using standard cooling procedures. The cooled emulsion was verified by temperature using a suitable temperature meter, for example, Cooper-Atkins temperature probe (Model: DPP400W). Once the microemulsion cooled down acid modifies, like citric acid, sugar alcohols, preservatives, sugars, sweeteners or flavoring agents can be added as desired for the finished product. The other ingredients are added to the microemulsion, and mixed at 400 RPM for 20 minutes. The pH of the microemulsion was verified to range between 2.6 and 3.4. The microemulsion can be optionally filtered using a standard 200-micron end-product filter, or another standard filter that follows good manufacturers' practices (GMP) before further evaluation, dilution or use.

Table 1C, 1D, and 1E sets forth ingredient compositions of other cationic microemulsions that may be prepared using ethyl lauroyl arginate with mixtures of polysorbate 80 and TPGS, and mixtures of polysorbate 60 and polysorbate 80.

Table 1F sets forth ingredient compositions of the diluted master stock microemulsions 1B, 1C, or 1D. The dilution can be done in a suitable GMP-certified stainless-steel tank. This may be modified to align with the desired beverage target for the finished consumer packaged good product. The dilution of either master stock microemulsion 1B, 1C, or 1D could follow the compositions set forth in Table 1F. After the master stock microemulsion 1B is diluted with water according to Table 1F, addition of ethyl lauroyl arginate are added. As desired in the finish product, sugar alcohols, preservatives, sugars, sweeteners and flavoring agents can be added. The final batch solution is mixed at 400 RPM using standard GMP-certified mixers for 15 minutes. This diluted microemulsion can be optionally filtered using a standard 200-micron end-product filter, or another standard filter that follows GMP standards before further use. This diluted microemulsion may be bottled into standard amber glass bottles or aluminum cans known in the industry from prior art using standard bottle packing equipment known in the industry from prior art.

Alternatively, the TPGS, polysorbate 80 and/or polysorbate 60 can be replaced with polyglyceryl esters of fatty acids. Polyglyceryl esters of fatty acids includes polyglyceryl monoesters and polyglyceryl multiesters where polyglyceryl monoesters are selected from the group consisting of Polyglyceryl-3 stearate, Polyglyceryl-3 oleate, Polyglyceryl-3 laurate, Polyglyceryl-3 caprylate, Polyglyceryl-3 palmitate, Polyglyceryl-3 myristate, Polyglyceryl-10 Hydroxystearate, Polyglyceryl-4 Caprate, Polyglyceryl-4 Caprylate, Polyglyceryl-10 Eicosadioate, Polyglyceryl-10 Behenate, Polyglyceryl-4 Laurate, Polyglyceryl-4 Isostearate, Polyglyceryl-10 Oleate, Polyglyceryl-10 Stearate, Polyglyceryl-4 Oleate, Polyglyceryl-5 Laurate, Polyglyceryl-8 Oleate, Polyglyceryl-8 Stearate, Polyglyceryl-10 Palmitate, Polyglyceryl-10 Isostearate, Polyglyceryl-10 Laurate, Polyglyceryl-10 Myristate, Polyglyceryl-5 Myristate, Polyglyceryl-5 Isostearate, Polyglyceryl-5 Stearate, Polyglyceryl-6 Isostearate, Polyglyceryl-6 Stearate, Polyglyceryl-5 Oleate, and Polyglyceryl-6 Oleate; wherein the polyglyceryl multiester is selected from the group consisting of Polyglyceryl-10 fatty ester (POLYALDO® 10-2-P), Polyglyceryl-10 Caprylate/Caprate and Polyglyceryl-10 Heptahydroxystearate, Polyglyceryl-5 Triisostearate, Polyglyceryl-5 Dioleate, Polyglyceryl-10 Pentastearate, Polyglyceryl-10 Pentahydroxystearate, Polyglyceryl-5 Trioleate, Polyglyceryl-6 Tricaprylate, Polyglyceryl-10 Distearate, Polyglyceryl-10 Tristearate, Polyglyceryl-6 Dioleate, Polyglyceryl-6 Distearate, Polyglyceryl-10 Pentaoleate, Polyglyceryl-10 Decaoleate, Polyglyceryl-6 Pentastearate, Polyglyceryl-6 Octastearate, Polyglyceryl-10 Decaisostearate, Polyglyceryl-10 Dioleate, Polyglyceryl-8 Decaerucate, Polyglyceryl-8 Decaisostearate, Polyglyceryl-10 Pentaisostearate, Polyglyceryl-10 Nonaisostearate, Polyglyceryl-10 Dipalmitate, Polyglyceryl-8 Decaricinoleate, and Polyglyceryl-10 Diisostearate.

The novelty in these cationic microemulsions is that the disclosed microemulsions containing ethyl lauroyl arginate will have a positive surface charge. The positive surface charge can be measured by appropriate equipment, such as zeta potential measurements typically performed by a dynamic light scattering equipment. This charge is opposite to the conventional microemulsions that are common from prior art and currently in the industry where the microemulsions are prepared with a negative surface charge. The cationic ingredient, ethyl lauroyl arginate, could also benefit the microemulsions by compacting the microemulsions into a smaller diameter. Scientific literature supports the idea that positively charge emulsions, and microemulsions, can better interact with human cells, especially the cells that line the intestinal wall. This better interaction of the cationic microemulsions with the intestinal cells will lead to higher bioavailability resulting in more of the cannabinoid, THC, CBD, or other cannabinoids, being delivered into our blood. Conventional microemulsions that are negatively charged are repulsed from the negatively charged intestinal walls and the negative-negative charge repulsion is a barrier to optimal delivery of lipophilic components. The benefits of cationic microemulsions are on top of the common knowledge of ethyl lauroyl arginate being used as a preservative. The added preservative will then lead to longer shelf-life stability of the microemulsions as well as the finished packaged good.

TABLE 1A
Microemulsion compositions

| Function | Ingredient | w/w % |
|---|---|---|
| Lipophile | Any Lipophile | 0.01-10.00 |
| Emulsifier 1 | TPGS | 0-25 |
| Emulsifier 2 | Polysorbate 80 | 0-40 |
| Emulsifier 3 | Polysorbate 60 | 0-30 |
| Emulsifier 4 | Ethyl Lauroyl Arginate | 0.01-10.00 |
| Carrier Oil | Sunflower oil | 0.01-5.00 |
| Aqueous | Water | Remainder to 100 |

TABLE 1B
Microemulsion master stock with TPGS, polysorbate
80 and ethyl lauroyl arginate

| Function | Ingredient | w/w % | Mass (kg) |
|---|---|---|---|
| Lipophile | Cannabis distillate | 1.6 | 0.8 |
| Emulsifier 1 | TPGS or Polysorbate 80 | 20 | 10 |
| Emulsifier 3 | Ethyl Lauroyl Arginate | 1 | 0.5 |
| Carrier Oil | Sunflower oil | 1.9 | 0.95 |
| Aqueous | Water | 75.5 | 37.75 |

| | | |
|---|---|---|
| Total | 100 | 50 |

TABLE 1C
Microemulsion master stock with TPGS, polysorbate
80 and ethyl lauroyl arginate

| Function | Ingredient | w/w % | Mass (kg) |
|---|---|---|---|
| Lipophile | Cannabis distillate | 3.8 | 1.9 |
| Emulsifier 1 | TPGS | 20 | 10 |
| Emulsifier 2 | Polysorbate 80 | 20 | 10 |
| Emulsifier 3 | Ethyl Lauroyl Arginate | 1 | 0.5 |
| Carrier Oil | Sunflower oil | 1.9 | 0.95 |
| Aqueous | Water | 53.3 | 26.65 |

| | | |
|---|---|---|
| Total | 100 | 50 |

TABLE 1D
Microemulsion master stock with polysorbate 80,
polysorbate 60, and ethyl lauroyl arginate

| Function | Ingredient | w/w % | Mass (kg) |
|---|---|---|---|
| Lipophile | Cannabis distillate | 1.60 | 0.95 |
| Emulsifier 1 | Polysorbate 80 | 24.80 | 12.40 |
| Emulsifier 2 | Polysorbate 60 | 1.00 | 0.50 |
| Emulsifier 3 | Ethyl Lauroyl Arginate | 1.00 | 0.50 |
| Carrier Oil | Sunflower oil | 1.90 | 0.95 |
| Aqueous | Water | 69.71 | 34.70 |

| | | |
|---|---|---|
| Total | 100.00 | 50.00 |

TABLE 1F
Dilution into Beverage compositions

| Function | Ingredient | w/w % |
|---|---|---|
| Microemulsion | Microemulsion 1B, 1C or 1D | 0.001-10.000 |
| Master Stock | | |
| Emulsifier 3 | Ethyl Lauroyl Arginate | 0.001-0.100 |
| Aqueous | Water | Remainer to 100 |

### Example 2: Cationic Nanoemulsions Using Ethyl Lauroyl Arginate

Table 2A below depicts the nanoemulsion composition ranges that are covered by this disclosure. The label for “any lipophile” may include either one lipophile or a combination of two or more lipophiles previously described. Tables 2B, 2C, 2D, and 2E depict the nanoemulsion compositions of the ingredients required to make a 50 kg batch of cationic nanoemulsions containing 1.267% of cannabis distillate with 80% purity for THC. It should be noted that the ingredients in the nanoemulsion are added with an additional 5 weight % overage. Water is described as sterile distilled water. The cannabis distillate may be obtained from various standard processing pathways obtained through either supercritical or subcritical carbon dioxide extraction. Cannabis distillate may also be replaced with cannabis extract obtained from ethanol, propane, or butane extraction. Exemplary food grade Polysorbate 80 was sourced from Spectrum Chemical Mfg Corp (product number: P0138, Cas. Number: 9005-65-6). Exemplary food grade ethyl lauroyl arginate was sourced from Vedeqsa (Product name: Miranat-G, Product codeL 3407). Exemplary food grade sunflower oil is sourced from Jedwards Internation Inc (SKU: 51630). Sugar alcohol, preservatives, sugars, sweeteners and flavoring agents may be added to each table as desired in the finished consumer packaged good product.

Table 2B, 2C, 2D and 2E below sets forth ingredient compositions that would be compatible with the preparation of the cationic nanoemulsions. Table 2F sets forth ingredients that are required for a 50 kg batch of master stock nanoemulsion 2B containing polysorbate 80 and ethyl lauroyl arginate. This assumes that the cannabis distillate has 80% purity for THC. The ingredients are added into a suitable stainless-steel hot liquid tank. Exemplary of the tanks that can be used with the provided methods are water-jacketed tanks, for example, the Camak 42-gallon water-jacketed tank (Model: AGM 90 S 4). An appropriate scale is used to accurately measure the mass of each ingredient being added into the water-jacketed tank. The water is then heated to 90° C. (+5° C.) and the batch is mixed at 400 RPM for 30 minutes. The temperature was monitored with a suitable temperature meter, for example, Cooper-Atkins temperature probe (Model: DPP400W). After the intended mixing and heating time, the nanoemulsion was cooled down to 25° C. (+5° C.). using standard cooling procedures. The cooled emulsion was verified by temperature using a suitable temperature meter, for example, Cooper-Atkins temperature probe (Model: DPP400W). The nanoemulsion is then processed twice through a suitable microfluidizer. One example would include the Microfluidics Corps microfluidizer (Model: M-110EH) using a pressure of at least 17,000 PSI under maximum flow rate. Alternatively, the nanoemulsion may be processed through a suitable ultrasonicator. One example of an ultrasonicator would be the Industrial Sonomechanics ultrasonicator (model: ISP-3000). Another processing alternative includes the use of homogeniziers. One example of a homogenizer includes high shear mixers that comprise of rotor stators. Once the nanoemulsion is processed through a microfluidizer or ultrasonicator, acid modifiers, like citric acid, sugar alcohols, preservatives, sugars, sweeteners or flavoring agents can be added as desired for the finished product. The other ingredients are added to the nanoemulsion, and mixed at 400 RPM for 20 minutes. The nanoemulsion can be optionally filtered using a standard 200-micron end-product filter, or another standard filter that follow GMP standards before further evaluation, dilution, or use. This master stock nanoemulsion may be bottled into standard amber glass bottles using standard bottle packing equipment known in the industry.

Table 2F sets forth ingredient compositions of the diluted master stock nanoemulsions of either 2B, 2C, 2D, or 2E. The dilution can be done in a suitable GMP-certified stainless-steel tank. This may be modified to align with the desired beverage target for the finished consumer packaged good product. After the master stock nanoemulsion from Table 2B, 2C, 2D, or 2E is diluted with water according to Table 2F, addition of ethyl lauroyl arginate, acid modifiers, like citric acid, sugar alcohols, preservatives, sugars, sweeteners or flavoring agents are added. The final batch solution is mixed at 400 RPM using standard GMP-certified mixers for 15 minutes. This diluted nanoemulsion can be optionally filtered using a standard 200-micron end-product filter, or another standard filter that follows good manufacturers' practices before further use. This diluted nanoemulsion may be bottled into standard amber glass bottles or aluminum cans known in the industry from prior art using standard bottle packing equipment known in the industry from prior art.

Alternatively, the TPGS, polysorbate 80 and/or polysorbate 60 can be replaced with polyglyceryl esters of fatty acids. Polyglyceryl esters of fatty acids includes polyglyceryl monoesters and polyglyceryl multiesters where polyglyceryl monoesters are selected from the group consisting of Polyglyceryl-3 stearate, Polyglyceryl-3 oleate, Polyglyceryl-3 laurate, Polyglyceryl-3 caprylate, Polyglyceryl-3 palmitate, Polyglyceryl-3 myristate, Polyglyceryl-10 Hydroxystearate, Polyglyceryl-4 Caprate, Polyglyceryl-4 Caprylate, Polyglyceryl-10 Eicosadioate, Polyglyceryl-10 Behenate, Polyglyceryl-4 Laurate, Polyglyceryl-4 Isostearate, Polyglyceryl-10 Oleate, Polyglyceryl-10 Stearate, Polyglyceryl-4 Oleate, Polyglyceryl-5 Laurate, Polyglyceryl-8 Oleate, Polyglyceryl-8 Stearate, Polyglyceryl-10 Palmitate, Polyglyceryl-10 Isostearate, Polyglyceryl-10 Laurate, Polyglyceryl-10 Myristate, Polyglyceryl-5 Myristate, Polyglyceryl-5 Isostearate, Polyglyceryl-5 Stearate, Polyglyceryl-6 Isostearate, Polyglyceryl-6 Stearate, Polyglyceryl-5 Oleate, and Polyglyceryl-6 Oleate; wherein the polyglyceryl multiester is selected from the group consisting of Polyglyceryl-10 fatty ester (POLYALDO® 10-2-P), Polyglyceryl-10 Caprylate/Caprate and Polyglyceryl-10 Heptahydroxystearate, Polyglyceryl-5 Triisostearate, Polyglyceryl-5 Dioleate, Polyglyceryl-10 Pentastearate, Polyglyceryl-10 Pentahydroxystearate, Polyglyceryl-5 Trioleate, Polyglyceryl-6 Tricaprylate, Polyglyceryl-10 Distearate, Polyglyceryl-10 Tristearate, Polyglyceryl-6 Dioleate, Polyglyceryl-6 Distearate, Polyglyceryl-10 Pentaoleate, Polyglyceryl-10 Decaoleate, Polyglyceryl-6 Pentastearate, Polyglyceryl-6 Octastearate, Polyglyceryl-10 Decaisostearate, Polyglyceryl-10 Dioleate, Polyglyceryl-8 Decaerucate, Polyglyceryl-8 Decaisostearate, Polyglyceryl-10 Pentaisostearate, Polyglyceryl-10 Nonaisostearate, Polyglyceryl-10 Dipalmitate, Polyglyceryl-8 Decaricinoleate, and Polyglyceryl-10 Diisostearate.

The novelty in these cationic nanoemulsions is that the disclosed nanoemulsions containing ethyl lauroyl arginate will have a positive surface charge. The positive surface charge can be measured by appropriate equipment, such as zeta potential measurements typically performed by a dynamic light scattering equipment. This charge is opposite to the conventional nanoemulsions that are common from prior art and currently in the industry where the nanoemulsions are prepared with a negative surface charge. The cationic ingredient, ethyl lauroyl arginate, could also benefit the nanoemulsions by compacting the nanoemulsions into a smaller diameter. Scientific literature supports the idea that positively charge emulsions, and nanoemulsions, can better interact with human cells, especially the cells that line the intestinal wall. This better interaction of the cationic nanoemulsions with the intestinal cells will lead to higher bioavailability resulting in more of the cannabinoid, THC, CBD, or other cannabinoids, being delivered into our blood. Conventional nanoemulsions that are negatively charged are repulsed from the negatively charged intestinal walls and the negative-negative charge repulsion is a barrier to optimal delivery of lipophilic components. The benefits of cationic nanoemulsions are on top of the common knowledge of ethyl lauroyl arginate being used as a preservative. The added preservative will then lead to longer shelf-life stability of the nanoemulsions as well as the finished packaged good.

TABLE 2A
Nanoemulsion containing polysorbate 80, lecithin,
and ethyl lauroyl arginate compositions

| Function | Ingredient | w/w % |
|---|---|---|
| Lipophile | Any Lipophile | 0.01-10.00 |
| Emulsifier 1 | Polysorbate 80 | 1-10 |
| Emulsifier 2 | Lecithin | 0-10 |
| Emulsifier 3 | Polysorbate 60 | 0-10 |
| Emulsifier 4 | Sucrose monopalmitate | 0-10 |
| Emulsifier 5 | Ethyl Lauroyl Arginate | 0.01-10.00 |
| Carrier Oil | Sunflower oil | 0.01-5.00 |
| Aqueous | Water | Remainder to 100 |

TABLE 2B
Nanoemulsion master stock with polysorbate
80, lecithin, and ethyl lauroyl arginate

| Function | Ingredient | w/w % | Mass (kg) |
|---|---|---|---|
| Lipophile | Cannabis distillate | 1.267 | 0.633 |
| Emulsifier 1 | Polysorbate 80 | 2.533 | 1.267 |
| Emulsifier 3 | Ethyl Lauroyl Arginate | 1.000 | 0.500 |
| Carrier Oil | Sunflower oil | 0.634 | 0.317 |
| Aqueous | Water | 94.567 | 47.283 |

| | | |
|---|---|---|
| Total | 100 | 50 |

TABLE 2C
Nanoemulsion master stock with polysorbate
80, lecithin, and ethyl lauroyl arginate

| Function | Ingredient | w/w % | Mass (kg) |
|---|---|---|---|
| Lipophile | Cannabis distillate | 1.267 | 0.633 |
| Emulsifier 1 | Polysorbate 80 | 2.533 | 1.267 |
| Emulsifier 2 | Lecithin | 2.533 | 1.267 |
| Emulsifier 3 | Ethyl Lauroyl Arginate | 1 | 0.5 |
| Carrier Oil | Sunflower oil | 0.6335 | 0.31675 |
| Aqueous | Water | 93.3005 | 46.64925 |

| | | |
|---|---|---|
| Total | 100 | 50 |

TABLE 2D
Nanoemulsion master stock with polysorbate 80, polysorbate
60, lecithin and ethyl lauroyl arginate

| Function | Ingredient | w/w % | Mass (kg) |
|---|---|---|---|
| Lipophile | Cannabis distillate | 1.267 | 0.633 |
| Emulsifier 1 | Polysorbate 80 | 2.533 | 1.267 |
| Emulsifier 2 | Polysorbate 60 | 2.533 | 1.267 |
| Emulsifier 3 | Lecithin | 2.533 | 1.267 |
| Emulsifier 4 | Ethyl Lauroyl Arginate | 1 | 0.5 |
| Carrier Oil | Sunflower oil | 0.6335 | 0.31675 |
| Aqueous | Water | 93.3005 | 46.64925 |

| | | |
|---|---|---|
| Total | 100 | 50 |

TABLE 2E
Nanoemulsion master stock with polysorbate 80, polysorbate 60,
sucrose monopalmitate, lecithin and ethyl lauroyl arginate

| Function | Ingredient | w/w % | Mass (kg) |
|---|---|---|---|
| Lipophile | Cannabis distillate | 1.267 | 0.633 |
| Emulsifier 1 | Polysorbate 80 | 2.533 | 1.267 |
| Emulsifier 2 | Polysorbate 60 | 2.533 | 1.267 |
| Emulsifier 3 | Sucrose monopalmitate | 0.7 | 0.35 |
| Emulsifier 4 | Lecithin | 2.533 | 1.267 |
| Emulsifier 5 | Ethyl Lauroyl Arginate | 1 | 0.5 |
| Carrier Oil | Sunflower oil | 0.6335 | 0.31675 |
| Aqueous | Water | 88.8005 | 44.39925 |

| | | |
|---|---|---|
| Total | 100 | 50 |

TABLE 2F
Dilution into Beverage Compositions

| Function | Ingredient | w/w % |
|---|---|---|
| Nanoemulsion | Nanoemulsion 2B, 2C, 2D or 2E | 0.001-5.000 |
| Master Stock | | |
| Emulsifier 3 | Ethyl Lauroyl Arginate | 0.001-0.100 |
| Aqueous | Water | Remainder to 100 |

What is claimed is:

1. A cationic emulsion composition comprising:
at least one lipophile in an amount between 0.01% and 10% by weight (w/w) of the composition;
at least one ingestible emulsifier in an amount between 1% and 40% w/w;
an ingestible cationic emulsifier in an amount between 0.01% and 10%;
an ingestible oil in an amount between 0.01% and 5%;
an ingestible pH adjuster in an amount between 0.001% and 5%; and
an ingestible preservative in an amount between 0.0001% and 1%.

2. (canceled)

3. (canceled)

4. (canceled)

5. The cationic emulsion of claim 1, wherein the phytocannabinoid comprises: Tetrahydrocannabinolic acid A (THCA-A), Tetrahydrocannabinolic acid B (THCA-B), Tetrahydrocannabinol (THC), Tetrahydrocannabinolic acid C (THCA-C), Tetrahydrocannbinol C (THC-C), Tetrahydrocannabi varinic acid (THCVA), Tetrahydrocannabivarin (THCV), Tetrahydrocannabiorcolic acid (THCA-C), Tetrahydrocannabiorcol (THC-C), Delta-7-cis-iso-tetrahydrocannabi varin, A-tetrahydrocannabinolic acid (A8-THCA), A-tetrahydrocannabinol (A-THC), Cannabidiolic Acid (CBDA), Cannabidiol (CBD), Cannabidiol monomethyl ether (CBDM), Cannabidiol-C(CBD-C), Cannabidivarinic Acid (CBDVA), Cannabidivarin (CBDV), Cannabidiorcol (CBD-C), Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CB CVA), Cannabichromevarin (CBCV), Cannabicyclolic acid (CBLA), Cannabicyclol (CBL), Cannabicyclovarin (CBLV), Cannabielsoic add A (CBEA-A), Cannabielsoic add B (CBEA-B), Cannabielsoin (CBE), Cannabinolic add (CBNA), Cannabinol (CBN), Cannabinol methylether (CBNM), Cannabinol-C(CBN-C), Cannabivarin (CBV), Cannabino-C(CBN-C), Cannabiorcol (CBN-C), Cannabinodiol (CBND), Cannabinodivarin (CBDV), Cannabitriol (CBT), 10-Ethoxy-9-hydroxy-A&quot;-tetrahydrocannabinol, 8.9-Dihydroxy-A& #39; -tetrahydrocannabinol (8.9-Di-OH CBT-C), Cannabitriolvarin (CBTV), Ethoxy-cannabitriol varin (CBTVE), Dehydrocannabifuran (DCBF), Cannbifuran (CBF), Cannabichromanon (CBCN), Cannabicitran (CBT), 1O-Oxo-A& #39; -tetrahydrocannabinol (OTHC), A-cis-tetrahydrocannabinol (cis-THC), Cannabiripsol (CBR), 3,4,5,6-tetrahydro-7-hydroxy-alpha-alpha-2-trim, ethyl-9-n-propyl-2,6-methano-2H-1-benzoxocin-5-methanol (OH-iso-HHCV), Trihydroxy-delta-9-tetrahydrocannabinol (triCH-THC), Isocanabinoids, Epigallocatechin gallate or combinations thereof.

6. The cationic emulsion of claim 1, wherein the lipophile comprises: terpenes such as β-Myrcene, D-Limonene, β-Ocimene, γ-Terpinene, α-Terpinene, α-Terpineol, α-Pinene, β-Pinene, Linalool, Camphene, Terpinolene, α-Phellandrene, γ-Cadinene, Δ3-Carene, ρ-Cymene, fenchol, 1, 8-cineole (eucalyptol), β-Caryophyllene, Caryophyllene Oxide, Humulene, (α-Caryophyllene), β-Elemene, Guaiol, Eudesmol Isomers, Nerolidol, Gurjunene, γ-Cadinene, β-Farnesene, carvone, taxadiene, squalene, geraniol, geranylfarnesol, lycopene or mixtures thereof; or antioxidants, fat soluble vitamins, fatty acids, carotenoids, hormones, metabolic factors, phytochemicals, phytonutrients, phytosterols and vitamin derivatives.

7. The cationic emulsion composition of claim 1, wherein the ingestible emulsifier comprises at least one of tocopheryl polyethylene glycol succinate (TPGS 1000), and polysorbate 80.

8. (canceled)

9. The cationic emulsion composition of claim 1, wherein the ingestible cationic emulsifier comprises ethyl lauroyl arginate alone or in combination with either propylene glycol or glycerol.

10. (canceled)

11. (canceled)

12. The cationic emulsion composition of 1, wherein the ingestible preservative comprises at least one preservative selected from the group of vitamin e, vitamin c, sulphurous acid, sorbic acid, calcium sorbate, erythorbic acid, sodium ascorbate, calcium ascorbate, benzoic acid, sodium benzoate, at least one potassium sorbate, sodium bisulphite, or combinations thereof.

13. The cationic emulsion composition of claim 1, further comprising an ingestible co-solvent in an amount between 0.1% and 50% w/w.

14. The cationic emulsion composition of claim 1, wherein the ingestible co-solvent comprises propylene glycol, glycerin, ethanol, or a combination thereof.

15. (canceled)

16. (canceled)

17. (canceled)

18. (canceled)

19. (canceled)

20. The cationic emulsion composition of claim 1, further comprising: at least one oil selected from the group consisting of medium-chain triglycerides (MCTs), short-chain triglycerides, cannabis oil (hemp oil), coconut oil, cottonseed oil, soybean oil, almond oil, sesame oil, rapeseed oil, peppermint oil, canola oil, palm kernel oil, arachis oil, olive oil, sunflower oil, castor oil, corn oil, olive oil, palm oil, peanut oil, hydrogenated soybean oil, linseed oil, hazelnut oil, safflower oil, rice bran oil, or combinations thereof.

21. (canceled)

22. The cationic emulsion composition of claim 1, further comprising a reducing agent selected from the group consisting of vitamin C, ubiquinol, L-ascorbic acid-8-palmitate, or a combination thereof.

23. (canceled)

24. The cationic emulsion composition of claim 1, further comprising a zeta-potential between at or about +10 millivolts (mV) and at or about +60 mVs, inclusive, as measured by an electrophoretic mobility apparatus.

25. (canceled)

26. (canceled)

27. A method for the preparation of a cationic emulsion composition according to claim 1, comprising: weighing the components of the emulsion composition; heating the combined emulsion composition from about 751° C. to about 95° C. with agitation from a rotor/stator to prepare the microemulsion; and cooling the emulsion composition to about 25° C.

28. (canceled)

29. (canceled)

30. A method for the preparation of a cationic emulsion composition according to claim 1, comprising: weighing the components of the emulsion composition; heating the combined emulsion from about 75° C. to about 95° C. with agitation to prepare the microemulsion composition; cooling the nanoemulsion to about 75° C.; and passing the emulsion composition through an appropriate microfluidizer processor at least once with at least 4,000 pounds per square inch (PSI).

31. A method for the preparation of a cationic emulsion composition according to claim 1, comprising the steps of: weighing the components of the emulsion composition; heating the combined emulsion composition from about 75° C. to about 95° C. with agitation to prepare the emulsion composition; cooling the emulsion composition to about 25° C.; and passing the emulsion composition through an appropriate ultrasonic homogenizer that operates between 0.5 to 25 kW.

32. (canceled)

33. The cationic emulsion composition of claim 1 in which the composition is clear and does not contain visible particles or precipitants with a Nephelometric Turbidity Units (NTU) value of less than 200, or about 200.

34. The cationic emulsion composition of claim 1 in which the composition has particle sizes that are less than 600 nm, and less than 400 nm, or less than 250 nm, as measured by dynamic light scattering equipment.

35. The cationic emulsion composition of claim 1 intended for incorporation into a group consisting of topical ointments and creams, edibles, orally dissolvable films, soft chews, and dissolvable powders, comprising: providing a cationic emulsion composition; and diluting the cationic emulsion composition into the intended end formulation using appropriate GMP equipment and manufacturing processes.

36. The cationic emulsion composition of claim 1 wherein the ingestible emulsifiers comprise ingestible polyglyceryl monoesters or polyglyceryl multiesters.

37. (canceled)

38. The composition of claim 36, wherein the ingestible polyglyceryl monoester or polyglyceryl multiester is selected from the group consisting of Polyglyceryl-3 stearate, Polyglyceryl-3 oleate, Polyglyceryl-3 laurate, Polyglyceryl-3 caprylate, Polyglyceryl-3 palmitate, Polyglyceryl-3 myristate, Polyglyceryl-10 Hydroxystearate, Polyglyceryl-4 Caprate, Polyglyceryl-4 Caprylate, Polyglyceryl-10 Eicosadioate, Polyglyceryl-10 Behenate, Polyglyceryl-4 Laurate, Polyglyceryl-4 Isostearate, Polyglyceryl-10 Oleate, Polyglyceryl-10 Mono- and Dioleate, Polyglyceryl-10 Stearate, Polyglyceryl-4 Oleate, Polyglyceryl-5 Laurate, Polyglyceryl-8 Oleate, Polyglyceryl-8 Stearate, Polyglyceryl-10 Palmitate, Polyglyceryl-10 Isostearate, Polyglyceryl-10 Laurate, Polyglyceryl-10 Myristate, Polyglyceryl-5 Myristate, Polyglyceryl-5 Isostearate, Polyglyceryl-5 Stearate, Polyglyceryl-6 Isostearate, Polyglyceryl-6 Stearate, Polyglyceryl-5 Oleate, and Polyglyceryl-6 Oleate; wherein the polyglyceryl multiester is selected from the group consisting of Polyglyceryl-10 fatty ester (POLYALDO® 10-2-P), Polyglyceryl-10 Caprylate/Caprate and Polyglyceryl-10 Heptahydroxystearate, Polyglyceryl-5 Triisostearate, Polyglyceryl-5 Dioleate, Polyglyceryl-10 Pentastearate, Polyglyceryl-10 Pentahydroxystearate, Polyglyceryl-5 Trioleate, Polyglyceryl-6 Tricaprylate, Polyglyceryl-10 Distearate, Polyglyceryl-10 Tristearate, Polyglyceryl-6 Dioleate, Polyglyceryl-6 Distearate, Polyglyceryl-10 Pentaoleate, Polyglyceryl-10 Decaoleate, Polyglyceryl-6 Pentastearate, Polyglyceryl-6 Octastearate, Polyglyceryl-10 Decaisostearate, Polyglyceryl-10 Dioleate, Polyglyceryl-8 Decaerucate, Polyglyceryl-8 Decaisostearate, Polyglyceryl-10 Pentaisostearate, Polyglyceryl-10 Nonaisostearate, Polyglyceryl-10 Dipalmitate, Polyglyceryl-8 Decaricinoleate, and Polyglyceryl-10 Diisostearate.

39. (canceled)

40. (canceled)

41. The cationic emulsion composition of claim 1 wherein the cationic emulsion composition is added to a food or beverage prior to oral administration or is consumed directly.

42. (canceled)
